(19) United States
(12) Patent Application Publication
(10) Pub. No.: US 2021/0177601 A1
(43) Pub. Date: Jun. 17, 2021

(54) IMPLANT AND KIT FOR THE TREATMENT AND/OR BIOLOGICAL RECONSTRUCTION OF A BONE DEFECT

(71) Applicant: AESCULAP AG, Tuttlingen (DE)

(72) Inventors: Georg HETTICH, Tuttlingen (DE); Silke KÖNIG, Rottweil (DE); Wolfgang ABELE, Tuttlingen (DE)

(21) Appl. No.: 16/761,742

(22) PCT Filed: Oct. 26, 2018
(86) PCT No.: PCT/EP2018/079383

§ 371 Date: May 5, 2020

(30) Foreign Application Priority Data
Nov. 20, 2017 (DE) 102017220710.8

(51) Int. Cl. A61F 2/30; A61F 2/28; A61L 27/54; A61L 27/20; A61L 27/34; A61L 27/18; A61L 27/12; A61L 27/26

(52) U.S. Cl. CPC A61F 2/30734; A61F 2/2846; A61L 27/54; A61L 27/20; A61L 27/34; A61L 27/18; A61L 2300/236; A61L 27/26; A61F 2002/30784; A61F 2002/30062; A61F 2002/30738; A61L 2430/02; A61L 27/12

(57) ABSTRACT

An implant, in particular for the treatment and/or biological reconstruction of a bone defect, includes osteoconductive supporting bodies and a supporting sheath at least partially surrounding the osteoconductive supporting bodies. The sheath can be formed of a sheath material or can consist of a sheath material that is soluble in water or in water-containing liquids. A kit and method for the treatment and/or biological reconstruction of a bone defect can utilize a sheath for osteoconductive supporting bodies and a material for sheathing the osteoconductive supporting bodies.

### CROSS-REFERENCE TO RELATED APPLICATIONS

This application is the United States national phase entry of International Application No. PCT/EP2018/079383, filed Oct. 26, 2018, and claims the benefit of priority of German application No. 10 2017 2207 10.8, filed Nov. 20, 2017. The contents of International Application No. PCT/EP2018/079383 and German application No. 10 2017 2207 10.8 are incorporated by reference herein in their entireties.

### FIELD

The invention relates to an implant and a kit for the treatment and/or biological reconstruction of a bone defect, a sheath for osteoconductive supporting bodies, the use of a material that is soluble in water or in a water-containing liquid for producing a sheath for osteoconductive supporting bodies and a method for the treatment and/or biological reconstruction of a bone defect.

### BACKGROUND

Large bone defects on the acetabulum are a serious problem during revision surgery on the hip joint. Hip revision surgery has three main goals.

First, the original joint center is to be restored.

Furthermore, a stable fixation of an implant used to treat an acetabular defect is sought, a distinction being made between so-called primary stability and so-called secondary stability.

Primary stability is understood to mean the fixation of the implant based on friction or fastening elements, such as bone screws, in the first weeks after an operation. Secondary stability is understood to mean the fixation of the implant based on osseous growths. Secondary stability usually sets in about a month after a surgical procedure and can last up to several years.

Finally, a so-called biological reconstruction of the osseous defect is sought. A biological reconstruction is understood to mean the penetration and/or the reconstruction of a bone defect with the body's own bone.

Acetabular defects are referred to as “contained” defects if vital patient bone and an inserted hip socket completely enclose the defect. In contrast, acetabular defects are referred to as uncontained defects if an inserted hip socket does not completely enclose the defect, i.e. uncontained portions remain.

In practice, the so-called “Impaction Bone Grafting” technique has become established for the treatment of contained acetabular defects. In this technique, pieces of bone (cancellous bone) approximately 0.5 cm in size are prepared from an allogeneic bone material (femoral head) and compacted into the bone defect using a suitable instrument. Using this technique, the acetabular defect can be biologically reconstructed and the center of rotation restored. However, allogeneic bone chips pose a risk of infection despite thermal treatment. Another disadvantage is the cost of producing allogeneic bone chips, which have increased significantly due to increased regulatory requirements. After all, intra-operative handling is extremely complicated and time-consuming. The human femoral head, from which the bone chips are produced, must be thawed and crushed during the operation. The chips produced in this way are irregular in shape and some surgical experience is required to use the chips properly.

Another approach to treating bone defects is artificial bone replacement materials, especially those based on calcium phosphate materials. A disadvantage of artificial bone replacement materials is often their limited mechanical resilience, i.e. the primary stability that can be achieved with them is limited. Another disadvantage is the risk of the bone replacement materials being degraded or absorbed without bone growth and consequently without achieving secondary stability.

U.S. Pat. No. 8,562,613 B2 discloses a kit for the treatment of bone defects with a mixture of an osteoconductive material and an osteoinductive material, and a porous container.

The subject matter of EP 0 764 008 B1 is a device for use in the stabilization of a spinal movement segment with a flexible bag, wherein the bag can contain a biological filling material for promoting bone or fiber growth.

EP 1 408 888 B1 discloses a system for correcting vertebral compression fractures, which comprises a porous bag and a filling tool, the filling tool being designed to inject a bone filling material under pressure into the porous bag.

An implantable container is known from WO 2012/061024 A1, which contains at least partially demineralized and osteoinductive bone particles.

The publication “Bone Regeneration by the Combined Use of Tetrapod-Shaped Calcium Phosphate Granules with Basic Fibroblast Growth Factor-Binding Ion Complex Gel in Canine Segmental Radial Defects (J. Vet. Med. Sci. 76(7):955-961, 2014)” by Honnami et al. deals with a combination of tetrapod-shaped granules of alpha-tricalcium phosphate and an osteoinductive gel.

### SUMMARY

It is the object of the invention to provide an implant which is suitable for the treatment and/or biological reconstruction of a bone defect, in particular periprosthetic bone defect such as an acetabular defect, and which avoids or at least partially avoids the disadvantages mentioned above, in particular in connection with a hip revision operation. The implant should in particular optimize the achievement of primary stability and/or secondary stability and/or biological reconstruction and enable simple and low-complication handling.

Furthermore, it is an object of the invention to provide a kit for the treatment and/or biological reconstruction of a bone defect, a sheath for osteoconductive supporting bodies, the use of a material for producing such a sheath and a method for the treatment and/or biological reconstruction of a bone defect.

These objects are achieved by an implant and by a kit disclosed in the description, a sheath disclosed in the description, a use disclosed in the description and by a method for the treatment and/or biological reconstruction of a bone defect disclosed in the description.

According to a first aspect, the invention relates to an implant, preferably for the treatment and/or biological reconstruction, in particular lining and/or sealing and/or relining and/or at least partially filling, a bone defect.

For the purposes of the present invention, the implant can also be referred to as bone replacement material.

The implant comprises the following:
osteoconductive supporting bodies and
a sheath that at least partially, preferably completely, surrounds the osteoconductive supporting bodies.

The sheath is particularly characterized by the fact that it comprises a sheath material or consists of a sheath material that is soluble in water or in a water-containing liquid. Such a sheath material is also abbreviated below as a soluble sheath material.

The sheath can be a film, a fleece, a nonwoven material, a mesh or a textile fabric, in particular a woven, braided or knitted fabric, such as a knitted or crocheted mesh. The sheath can be produced or manufactured in particular by extrusion, by blow molding, by injection molding, by a compression molding process or by an additive process. The sheath can further comprise monofilament thread material and/or multifilament thread material or consist of monofilament thread material and/or multifilament thread material.

For the purposes of the present invention, the term “bone defect” shall be understood to mean a bone area affected by loss of bone tissue, in particular joint bone tissue, preferably hip or knee joint bone tissue, or bone area affected by vertebral body tissue, in particular joint bone area, preferably hip joint or knee joint bone area, or vertebral body area. The bone loss can be the result of a bone fracture, a bone trauma, a bone disease such as tumor disease or a surgical intervention/reintervention, especially a revision after total hip or knee arthroplasty. Preferably, for the purposes of the present invention, the term “bone defect” shall be understood to mean a periprosthetic bone defect, i.e. a bone area affected by periprosthetic bone tissue loss, in particular due to mechanical overload and/or wear-induced osteolysis and/or implant migration.

The bone defect is preferably a joint bone defect, in particular a knee joint bone defect or a hip joint bone defect, preferably an acetabular defect, in particular a contained or uncontained acetabular defect.

Furthermore, for the purposes of the present invention, the term “bone defect” can mean a human bone defect or an animal bone defect.

For the purposes of the present invention, the term “animal bone defect” shall be understood to mean the bone defect of a non-human mammal, such as, for example, a horse, cow, goat, sheep, pig or a rodent, such as, for example, a rabbit, rat or mouse.

For the purposes of the present invention, the term “supporting body” shall be understood to mean bodies, in particular regularly and/or irregularly molded bodies, which are designed to withstand forces normally occurring in a bone defect to be treated and/or biologically reconstructed without deformation or destruction, but at least without substantial deformation or destruction and therefore to take on load-bearing functions. For this reason, for the purposes of the present invention, the supporting bodies can also be referred to as osteoconductive, load-bearing supporting bodies.

For the purposes of the present invention, the term “osteoconductive” used in connection with the supporting bodies shall be understood to mean the ability of the supporting bodies to form a three-dimensional structure, in particular a lead structure, or a three-dimensional matrix, in particular a lead matrix, which promotes ingrowth of bone tissue, in particular new bone tissue.

For the purposes of the present invention, the term “degradable in vivo” relates to a substance or a material which can be metabolized in a human or animal body, in particular by the action of enzymes. The breakdown of the substance or material can proceed completely all the way to mineralization, i.e. the release of chemical elements and their incorporation into inorganic compounds, such as, for example, carbon dioxide and/or oxygen and/or ammonia, or remain at the level of non-degradable intermediate or transformation products.

For the purposes of the present invention, the term “animal body” shall be understood to mean the body of a non-human mammal, such as, for example, a horse, cow, goat, sheep, pig or rodent, such as a rabbit, rat or mouse.

For the purposes of the present invention, the term “absorbable in vivo” refers to a substance or a material which can be absorbed in a human or animal body by living cells or living tissue, such as, for example, kidneys, without degradation or significant degradation of the substance or material taking place.

For the purposes of the present invention, the term “sheath” shall be understood to mean a structure or construct which is designed to partially, preferably completely, surround or enclose (at least) the osteoconductive supporting bodies. For this purpose, the sheath preferably has a void which can be filled or is filled with (at least) the osteoconductive supporting bodies at least partially, preferably only partially.

For the purposes of the present invention, the term “sheath material” shall be understood to mean a material which is part of the sheath and in particular is involved in the construction of the sheath. Depending on its solubility in water or in a water-containing liquid, the sheath material can be the main component or even the exclusive component of the sheath or only a secondary component, in particular in the sense of an additive.

For the purposes of the present invention, the term “water-containing liquid” shall be understood to mean an aqueous liquid, i.e. a liquid which contains water, the liquid optionally containing further substances, such as salts, proteins, polysaccharides, lipids, blood components or mixtures thereof, and/or cells. Accordingly, the water-containing liquid for the purposes of the present invention can be, for example, an aqueous dispersion, an aqueous solution such as aqueous salt solution, an aqueous suspension or a body fluid. For the purposes of the present invention, the term “water-containing liquid” shall preferably be understood to mean an aqueous solution or a body fluid, such as, for example, blood and/or tissue fluid and/or lymph fluid. It is further preferred that the water-containing liquid for the purposes of the present invention is free from organic solvents.

The present invention is characterized in particular by the following advantages:
The sheath serves particularly advantageously as an insertion aid for the osteoconductive supporting bodies, without representing a barrier for ingrowing bone tissue and therefore for a biological reconstruction of the osseous defect. The soluble sheath material particularly advantageously creates the prerequisite that the sheath at least partially dissolves after the implant has been placed in a bone defect, so that, with a good healing process, ingrowing bone tissue can penetrate the osteoconductive supporting bodies, whereby the latter can be integrated into the bone. As a result, a biological reconstruction and consequently a reduction in the size of the osseous defect is possible even in the case of osteoconductive supporting bodies that cannot be degraded in vivo or can only be degraded very slowly or are absorbable in vivo.

In the event of missing or poor bone growth, the osteoconductive supporting bodies are preferably retained and thus contribute to secondary stability. Thus, secondary stability can preferably be ensured, regardless of whether or not there is a built-up of bone tissue in the osteoconductive supporting bodies after implantation. This is particularly advantageous with regard to the care of older patients, in whom bone growth often no longer takes place or only occurs slightly.
Another advantage of the sheath is that it fixes the osteoconductive supporting body in place. In this way, an uncontrolled introduction of the supporting bodies into the bone defect can be avoided in a particularly advantageous manner.
A further advantage of the sheath is, in particular, that the osteoconductive supporting body can be compacted, in particular impacted, without an uncontrolled distribution of the osteoconductive supporting body within the bone defect during compacting, in particular impacting.

The sheath also offers the advantage of simple and, in particular, quick handling during a surgical procedure.
Another advantage is that the osteoconductive supporting bodies, in particular in the form of a loose or isolated bulk material, can be converted into a compacted, preferably impacted (clamped or wedged) state and in this state they can function as a lead structure for the ingrowth of bone tissue, in particular new bone tissue.
Another advantage is that the implant can, particularly when the osteoconductive supporting bodies are in a compacted, in particular impacted, state, be loaded permanently and, above all, homogeneously. A homogeneous implant load is a basic requirement for bone growth.

For example, the implant with compacted, in particular impacted, osteoconductive supporting bodies can be permanently subjected to a pressure load of up to 5 MPa. A structure produced by compacting, in particular impacting, and constructed by the osteoconductive supporting bodies can, with particular advantage, have an elastic deformation, in particular from 5% to 15%, and a low modulus of elasticity, in particular from 50 MPa to 300 MPa.

The osteoconductive supporting bodies preferably have at least one size or dimension in a size range from 0.5 mm to 5 mm, in particular 0.1 mm to 3 mm, preferably 1 mm to 2 mm. The at least one size or dimension can be in particular the height and/or width (thickness) and/or length and/or the diameter, in particular the average diameter, of the osteoconductive supporting bodies.

In a further embodiment, the osteoconductive supporting bodies are moveable relative to one another, in particular are displaceable relative to one another.

In a further embodiment, the osteoconductive supporting bodies can be impacted, i.e. mutually clamped or mutually wedged.

In a further embodiment, the osteoconductive supporting bodies, are mutually clamped or mutually wedged.

In a further embodiment, the osteoconductive supporting bodies can, preferably by impacting, be converted into a three-dimensional structure or matrix, in particular having voids and/or spaces, or are present in such a structure or matrix. For the purposes of the present invention such a structure or matrix can also be referred to as an osteoconductive lead structure or osteoconductive lead matrix.

The voids or spaces in the structure or matrix can have a diameter of 0.1 mm to 1.2 mm, in particular 0.2 mm to 1 mm, preferably 0.3 mm to 0.8 mm.
In contrast to generic implants, in particular metallic augmentations, in which only spaces at the implant edge zones are mechanically stressed, existing voids and/or spaces present in a compacted state of the osteoconductive supporting bodies within a complete defect filling, i.e. homogeneous, can be mechanically loaded (micro movements) due to a low modulus of elasticity. This in turn stimulates and/or reinforces bone growth, in particular new bone formation. Overall, a homogeneous ossification of the entire bone defect can be achieved.

In a configuration of the invention, the solubility of the sheath material is based on a physical dissolution process, i.e. the sheath material is soluble in water or in a water-containing liquid without degradation or other destruction.

In a further configuration of the invention, the solubility of the sheath material is based on degradation, in particular hydrolysis, such as, for example, enzyme-catalyzed hydrolysis, of the soluble sheath material.

In a further configuration of the invention, the soluble sheath material is soluble in water or in a water-containing liquid, in particular body fluid, for a period of 1 second to 72 hours, in particular 1 minute to 1 hour, preferably 2 minutes to 20 minutes.

In a further configuration of the invention, the soluble sheath material comprises or consists of a polysaccharide. The polysaccharide can be in particular a mucopolysaccharide and/or a cellulose derivative, in particular an alkyl cellulose and/or hydroxyalkyl cellulose. The polysaccharide is preferably selected from the group consisting of starch, amylose, amylopectin, dextran, methyl cellulose, hydroxymethyl cellulose, ethyl cellulose, hydroxyethyl cellulose, propyl cellulose, hydroxypropyl cellulose, butyl cellulose, hydroxybutyl cellulose, hydroxyethylmethyl cellulose, hydroxypropylmethyl cellulose, carboxymethyl cellulose, hyaluronic acid, chondroitin-4-sulfate, chondroitin-6-sulfate, keratin sulfate, alginic acid, heparin, heparan sulfate, chitosan, salts thereof or mixtures thereof.

In a further configuration of the invention, the soluble sheath material comprises a synthetic polymer or consists of such a polymer. The synthetic polymer is preferably selected from the group consisting of polyvinyl alcohol, polyethylene glycol, ethylene oxide-propylene oxide copolymers (EO-PO copolymers), ethylene oxide-propylene oxide block copolymers (EO-PO block copolymers), acrylic acid homopolymers, acrylic acid copolymers, polyvinylpyrrolidone homopolymers, polyvinylpyrrolidone copolymers or mixtures thereof.

The polyvinyl alcohol mentioned in the previous paragraph can be in particular a so-called low molecular weight polyvinyl alcohol, i.e. a polyvinyl alcohol with an average molecular weight of 10,000 to 30,000 and/or a so-called high molecular weight polyvinyl alcohol, i.e. a polyvinyl alcohol with an average molecular weight of 50,000 to 250,000.

In a further configuration of the invention, the soluble sheath material comprises a low molecular weight polyvinyl alcohol, i.e. a polyvinyl alcohol with an average molecular weight of 10,000 to 30,000 and/or a high molecular weight polyvinyl alcohol, i.e. a polyvinyl alcohol with an average molecular weight of 50,000 to 250,000, and also a polysaccharide, in particular a carboxyalkyl cellulose, preferably carboxymethyl cellulose.

In a further configuration of the invention, the soluble sheath material comprises a mixture of a polysaccharide and a synthetic polymer or consists of such a mixture. With regard to a suitable polysaccharide and synthetic polymer, reference is made to the polysaccharides and synthetic polymers described in the previous paragraphs.

The soluble sheath material can furthermore have a proportion of 0.1% by weight to 100% by weight, in particular 5% by weight to 100% by weight, preferably 90% by weight to 100% by weight, based on the total weight of the sheath. The higher the proportion of the soluble sheath material, the more completely the sheath can be dissolved in water or in a water-containing liquid. The solubility of the sheath can thus be controlled with particular advantage via the proportion of the soluble sheath material.

In a further configuration of the invention, the soluble sheath material is at least partially, in particular only partially or completely, crosslinked. The soluble sheath material can be chemically and/or physically cross-linked. For example, the sheath material can be crosslinked by a chemical crosslinking agent, in particular selected from the group consisting of formaldehyde, dialdehyde such as glutaraldehyde, polyaldehydes, diisocyanates, dicarbodiimides or mixtures thereof. The chemical crosslinking agent can have a proportion of 0.1% by weight to 20% by weight, in particular 1% by weight to 10% by weight, preferably 2% by weight to 5% by weight, based on the total weight of the sheath. Alternatively or in combination, the sheath material can be crosslinked by radiation, in particular gamma radiation, and/or by freeze-thaw cycles. Crosslinking, in particular the type and/or the degree of crosslinking, or a lack of crosslinking, can also be used to control the solubility of the sheath material and thus the sheath in water or in a water-containing liquid with particular advantage, with crosslinking generally reducing the solubility or increasing in the dissolution time.

In a further configuration of the invention, the sheath is free of a crosslinking agent.

In a further configuration of the invention, the sheath further comprises a sheath material which is insoluble in water or in a water-containing liquid. Such a sheath material is also abbreviated below as an insoluble sheath material.

The insoluble sheath material is preferably a polymer, especially a hydrophobic polymer.

The polymer is preferably selected from the group consisting of polyolefin, polyester, polyamide, polyurethane such as thermoplastic polyurethane, elastomer, polyimide, polyether ketone, polyether ether ketone, fluorocarbon, perfluorocarbon or mixtures thereof.

The polyolefin can be selected from the group consisting of polyethylene, polypropylene, polyacrylate, polymethacrylate, polymethyl methacrylate, polyvinylidene chloride, polyvinylidene fluoride, polytetrafluoroethylene, polytetrafluoropropylene, polyhexafluoropropylene, copolymers thereof or mixtures thereof.

The polyethylene may be in particular low density polyethylene, medium density polyethylene, high density polyethylene, ultra high molecular weight polyethylene, a copolymer thereof or mixtures thereof. In particular, the polyester can be selected from the group consisting of polyethylene terephthalate, polypropylene terephthalate, polybutylene terephthalate, copolymers thereof or mixtures thereof.

In particular, the polyamide can be selected from the group consisting of polyamide 6 (polyamide from caprolactam), polyamide 46 (polyamide from tetramethylene diamine and adipic acid), polyamide 6.6 (polyamide from hexamethylene diamine and adipic acid), polyamide 69 (polyamide from hexamethylene diamine and azelaic acid), polyamide 6/12 (polyamide from hexamethylene diamine and dodecanedioic acid), polyamide 1010 (polyamide from 1,10-decanediamine and sebacic acid), polyamide 11 (polyamide from alpha-aminoundecanoic acid), polyamide 12 (polyamide from laurolactam), polyamide 1212 (polyamide from dodecanediamine and dodecanedioic acid), silk, copolymers thereof or mixtures thereof.

The insoluble sheath material can have a proportion of 0.1% by weight to 99% by weight, in particular 0.1% by weight to 50% by weight, preferably 0.1% by weight to 1% by weight, based on the total weight of the sheath. The solubility of the sheath can also be deliberately influenced by the proportion of an optionally present insoluble sheath material. A proportion of an insoluble sheath material reduces the solubility of the sheath overall.

Furthermore, the insoluble sheath material can be present, in particular, in the form of an additive of the sheath.

For the purposes of the present invention, the term “additive” shall be understood to mean an admixture which has a maximum proportion of 49% by weight, in particular 0.1 to 40% by weight, preferably 2% by weight to 20% by weight, based on the total weight of the sheath.

In a further configuration of the invention, the sheath is free of a sheath material that is insoluble in water or in a water-containing liquid.

In a further configuration of the invention, the sheath or a section of the sheath has a thickness of 10 μm to 1 mm, in particular 20 μm to 800 μm, preferably 30 μm to 300 μm. In particular, the sheath can have a uniform thickness or, as will be explained in more detail below, a non-uniform thickness, i.e. have an irregular thickness.

In a further configuration of the invention, the sheath or a section of the sheath is designed in the form of a film. In other words, in a further configuration of the invention, the sheath or a section thereof is in the form of a film. A film-shaped section of the sheath preferably comprises a sheath material that is soluble in water or in a water-containing liquid or consists of such a sheath material. Furthermore, it is preferred if a film-shaped section of the sheath is arranged facing the bone in the implanted state. With regard to suitable soluble sheath materials, reference is made to the description above.

For the purposes of the present invention, the term “film-shaped” or “film” used in the context of the sheath is intended to define a sheath that preferably has a thickness of 10 μm to 1 mm, in particular 20 μm to 800 μm, preferably 30 μm to 300 μm. Such a thin configuration of the sheath is particularly advantageous with regard to the quickest possible solubility of the sheath material and thus of the sheath.

In a further configuration of the invention, a section of the sheath is textile, in particular mesh-shaped, i.e. in the form of a mesh. The section preferably comprises a sheath material which is insoluble in water or in a water-containing liquid or consists of such a sheath material. Furthermore, it is preferred if, in the implanted state, the section is arranged facing an artificial joint socket, in particular an artificial hip joint socket. With regard to suitable insoluble sheath materials, reference is made to the description above.

For the purposes of the present invention, the term “mesh-shaped” or “mesh” shall be understood to mean the shape of a textile fabric or a textile fabric which preferably has regular meshes or openings, the meshes or openings, for example, can be rhombic, square or hexagonal. In particular, the term “mesh-shaped” or “mesh” shall be understood to mean the shape of a braided or knitted fabric or a braided or knitted fabric.

In a further configuration of the invention, the sheath has sections, in particular a first section and a second section, which are designed differently. This makes it possible in particular to achieve a directional and/or side-dependent solubility of the sheath in water or in a water-containing liquid.

In a further configuration of the invention, the sections differ from one another with regard to the solubility of the sheath in water or in a water-containing liquid and/or with regard to the thickness of the sheath and/or with regard to the structure of the sheath.

In a further configuration of the invention, the sections differ from one another with regard to the chemical composition, in particular with regard to crosslinking of the sheath material. With regard to the chemical composition and possible crosslinking, reference is made to the (soluble and insoluble) sheath materials described so far and to the crosslinking options described so far.

In particular, the sections can differ from one another with regard to the type of crosslinking and/or the degree of crosslinking. For example, the sections can differ from one another with regard to a chemical crosslinking agent and/or a physical crosslinking. With regard to suitable chemical crosslinking agents and physical crosslinking, reference is made to the previous statements made in connection with an optional crosslinking of the soluble sheath material.

In a further configuration of the invention, the first section comprises a sheath material that is better or faster soluble in water or in a water-containing liquid or consists of such a sheath material, than the second section. In the implanted state, the first section is preferably arranged facing the bone, whereas, in the implanted state, the second section of the sheath is arranged facing an artificial joint socket, in particular an artificial hip joint socket. As a result, it can be achieved in a particularly advantageous manner that the sheath initially only dissolves in the area with respect to which bone tissue can be expected to grow in, whereas the remaining part of the sheath is characterized by a comparatively higher resistance to water or a water-containing liquid, so that a partial integrity of the sheath can be maintained at least during an initial phase after implantation. With regard to suitable sheath materials, reference is made to the description above.

In a further configuration of the invention, the first section comprises a weaker crosslinked sheath material that is soluble in water or in a water-containing liquid or consists of such a sheath material, than the second section. In other words, in a further configuration of the invention, the first section comprises a sheath material which is soluble in water or in a water-containing liquid and has a lower degree of crosslinking, or consists of such a sheath material, than the second section. In the implanted state, the first section is preferably arranged facing the bone, whereas, in the implanted state, the second section of the sheath is arranged facing an artificial joint socket, in particular an artificial hip joint socket. With regard to suitable sheath materials, reference is made to the description above.

In a further configuration of the invention, the first section comprises a non-crosslinked sheath material that is soluble in water or in a water-containing liquid, or consists of such a sheath material, whereas the second section comprises a crosslinked sheath material that is soluble in water or in a water-containing liquid or consists of such a sheath material. In the implanted state, the first section is preferably arranged facing the bone, whereas, in the implanted state, the second section of the sheath is arranged facing an artificial joint socket, in particular an artificial hip joint socket. With regard to suitable sheath materials, reference is made to the description above.

In a further configuration of the invention, the first section comprises a higher proportion of a sheath material that is soluble in water or in a water-containing liquid, than the second section. In the implanted state, the first section is preferably arranged facing the bone, whereas, in the implanted state, the second section of the sheath is arranged facing an artificial joint socket, in particular an artificial hip joint socket. With regard to suitable sheath materials, reference is made to the description above.

In a further configuration of the invention, the first section comprises a sheath material that is soluble in water or in a water-containing liquid or consists of such a sheath material, whereas the second section comprises a sheath material that is insoluble in water or in a water-containing liquid or consists of such a sheath material. In the implanted state, the first section is preferably arranged facing the bone, whereas, in the implanted state, the second section of the sheath is arranged facing an artificial joint socket, in particular an artificial hip joint socket. With regard to suitable sheath materials, reference is made to the description above.

In a further configuration of the invention, the first section has a smaller thickness than the second section. In the implanted state, the first section is preferably arranged facing the bone, whereas, in the implanted state, the second section of the sheath is arranged facing an artificial joint socket, in particular an artificial hip joint socket.

In a further configuration of the invention, the first section is not textile-shaped, in particular film-shaped, and the second section is textile-shaped, in particular mesh-shaped, i.e. in the form of a mesh. In the implanted state the first section is preferably arranged facing the bone, whereas, in the implanted state, the second section of the sheath is arranged facing an artificial joint socket, in particular an artificial hip joint socket.

In a further configuration of the invention, the sheath is designed to be dimensionally stable, in particular adapted to a bone defect.

In a further configuration of the invention, the sheath is only partially filled with the osteoconductive supporting bodies. For example, a void volume defined by the sheath can only be filled to 50% to 80% with the osteoconductive supporting bodies. In this way, a sheath that can be shaped and can therefore be adapted to the bone defect to be treated and/or biologically reconstructed can be realized with particular advantage.

In a further configuration of the invention, the osteoconductive supporting bodies are isolated osteoconductive supporting bodies. In other words, in a further configuration of the invention, the osteoconductive supporting bodies are present in isolated form, i.e. in the form of individual supporting bodies, for example in the form of a bulk material, as will be explained in more detail below.

In a further configuration of the invention, the osteoconductive supporting bodies comprise apatite and/or tricalcium phosphate or consist of apatite and/or tricalcium phosphate. The osteoconductive supporting bodies can each comprise a proportion of apatite from 0.1% by weight to 100% by weight. Furthermore, the osteoconductive supporting bodies can each comprise a proportion of tricalcium phosphate from 0.1% by weight to 100% by weight.

The apatite is preferably an apatite that cannot be degraded in vivo or is not absorbable in vivo. As a result, sufficient secondary stability can be achieved even in patients for whom (sufficient) bone growth can no longer be expected. This is particularly advantageous with regard to the treatment of bone defects in older patients.

Alternatively, the apatite can be an apatite that can be degraded in vivo or is absorbable in vivo, preferably an apatite that is slowly degradable in vivo or slowly absorbable in vivo. In particular, the apatite can have an in vivo degradation time or an in vivo absorption time of 6 months to 30 years, in particular 1 year to 20 years, preferably 4 years to 10 years. The degradation or absorption times provided in this paragraph are particularly advantageous with regard to the treatment of patients with slow or even missing bone growth.

The apatite can also be present in particular in crystalline form. A high degree of strength can be achieved by a high crystallinity. Due to a low crystallinity, good and/or quick degradability can be achieved.

Furthermore, the apatite can be a microcrystalline apatite, i.e. an apatite with crystallites which have at least one size or dimension in the micrometer range, in particular in a range>0.5 μm, in particular 0.6 μm to 500 μm, preferably 0.6 μm to 100 μm. The at least one size or dimension can be the length and/or width (thickness or height) and/or the diameter, especially the average diameter, of the crystallites.

Basically, the apatite can also be a macrocrystalline apatite.

Furthermore, the apatite can be a nanocrystalline apatite, i.e. an apatite with crystallites which have at least one size or dimension in the nanometer range, in particular in a range from 0.1 nm to 500 nm, preferably 0.1 nm to 100 nm. The at least one size or dimension can be the length and/or width (thickness or height) and/or the diameter, especially the average diameter, of the crystallites.

Furthermore, the apatite can be present in an amorphous form. This enables particularly good and/or rapid absorption to be achieved.

Furthermore, the apatite can be a phase-pure apatite. The term “phase-pure” shall be understood to mean in particular phase-pure in the sense of a relevant regulation, preferably according to ASTM F1185.

Furthermore, the apatite can have a porosity of less than 50%, in particular less than 20%, preferably less than 15%. Due to a low porosity, high mechanical stability can be achieved.

Furthermore, the apatite cannot be porous.

Furthermore, the osteoconductive supporting bodies can comprise a mixture of porous apatites and/or apatites with different porosity and/or non-porous apatites or consist of such a mixture, in particular if the supporting bodies are produced by an additive manufacturing process.

Furthermore, the apatite can be naturally occurring apatite or an apatite obtained from natural apatite.

Furthermore, the apatite can be a synthetic, i.e. man-made or artificial apatite.

The apatite is preferably selected from the group consisting of hydroxylapatite, fluorapatite, chlorapatite, carbonate-fluorapatite or mixtures thereof.

Particularly preferably, the apatite is hydroxylapatite. For example, the hydroxyapatite can be a fully synthetic, nanocrystalline and phase-pure hydroxyapatite. Such a hydroxyapatite is commercially available, for example under the registered trademark Ostim®.

The apatite and the tricalcium phosphate can also be present as a biphasic mixture. The biphasic structure is advantageous because the bone cells can grow in and the material is gradually replaced. The apatite and/or the tricalcium phosphate and/or the biphasic calcium phosphate have a porosity of 1% to 50%, in particular 5% to 20%, preferably 10% to 15%.

Furthermore, the apatite can be a sintered apatite.

The sintered apatite is preferably selected from the group consisting of sintered hydroxyapatite, sintered fluorapatite, sintered chlorapatite, sintered carbonate-fluorapatite or mixtures thereof.

In a further configuration of the invention, the osteoconductive supporting bodies comprise tricalcium phosphate or consist of tricalcium phosphate.

The tricalcium phosphate is preferably a tricalcium phosphate that is not degradable in vivo or is not absorbable in vivo. As a result, sufficient secondary stability can be achieved even in patients for whom (sufficient) bone growth can no longer be expected. This is particularly advantageous with regard to the treatment of bone defects in older patients.

Alternatively, the tricalcium phosphate can be a tricalcium phosphate that is degradable in vivo or absorbable in vivo, preferably a tricalcium phosphate that is slowly degradable in vivo or slowly absorbable in vivo. In particular, the tricalcium phosphate can have an in vivo degradation time or an in vivo absorption time of 1 month to 15 years, in particular 6 months to 10 years, preferably 1 year to 5 years. The degradation or absorption times provided in this paragraph are particularly advantageous with regard to the treatment of patients with slow bone growth.

The tricalcium phosphate can also be present in crystalline form. A high degree of strength can be achieved by a high crystallinity. Due to a low crystallinity, good and/or quick degradability can be achieved. Preferably the tricalcium phosphate has a crystallinity from 50% to 99%, in particular 75% to 95%.

Furthermore, the tricalcium phosphate can be microcrystalline tricalcium phosphate, i.e. to tricalcium phosphate with crystallites which have at least one size or dimension in the micrometer range, in particular in a range>0.5 μm, in particular 0.6 μm to 500 μm, preferably 0.6 μm to 100 μm. The at least one size or dimension can be the length and/or width (thickness or height) and/or the diameter, especially the average diameter, of the crystallites.

Basically, the tricalcium phosphate can also be a macrocrystalline tricalcium phosphate.

Furthermore, the tricalcium phosphate can be nanocrystalline tricalcium phosphate, i.e. to tricalcium phosphate with crystallites which have at least one size or dimension in the nanometer range, in particular in a range from 0.1 nm to 500 nm, preferably 0.1 nm to 100 nm. The at least one size or dimension can be the length and/or width (thickness or height) and/or the diameter, especially the average diameter, of the crystallites.

Furthermore, the tricalcium phosphate can be present in an amorphous form. This enables particularly good and/or rapid absorption to be achieved.

Furthermore, the tricalcium phosphate can be a phase-pure tricalcium phosphate. The term “phase-pure” shall be understood to mean in particular phase-pure in the sense of a relevant regulation, preferably according to ASTM F1088.

Furthermore, the tricalcium phosphate can have a porosity of less than 50%, in particular less than 20%, preferably less than 15%.

Furthermore, the tricalcium phosphate cannot be porous.

Furthermore, the tricalcium phosphate can be a naturally occurring tricalcium phosphate or a tricalcium phosphate obtained from natural tricalcium phosphate.

Furthermore, the tricalcium phosphate can be a synthetic, i.e. man-made or artificial, tricalcium phosphate.

The tricalcium phosphate is preferably selected from the group consisting of alpha-tricalcium phosphate (α-TCP), beta-tricalcium phosphate (β-TCP) and a mixture of alpha-tricalcium phosphate and beta-tricalcium phosphate.

In particular, the tricalcium phosphate can be sintered tricalcium phosphate.

The sintered tricalcium phosphate is preferably selected from the group consisting of sintered alpha-tricalcium phosphate, sintered beta-tricalcium phosphate and a mixture of sintered alpha-tricalcium phosphate and sintered beta-tricalcium phosphate.

Furthermore, the osteoconductive supporting bodies can comprise apatite and tricalcium phosphate or consist of apatite and tricalcium phosphate. The osteoconductive supporting bodies preferably comprise hydroxylapatite and beta-tricalcium phosphate or consist of hydroxylapatite and beta-tricalcium phosphate. With regard to further features and advantages of the apatite and the tricalcium phosphate, reference is made to the preceding statements.

In a further configuration of the invention, the osteoconductive supporting bodies have a roughened surface. This allows bone tissue growth or adherence, in particular to an osteoconductive lead structure formed by the supporting body, to be optimized. For the purposes of the present invention, the term “roughening” shall be understood to mean in particular that a roughness of the surface is increased after the supporting bodies have been shaped, in particular in a manufacturing step provided therefor. Roughening can be done, for example, by etching, in particular using phosphoric acid. The supporting bodies preferably have a roughened surface, the roughness of which is increased by at least 10% compared to a non-roughened supporting body surface. The term “roughness” shall be understood to mean in particular an unevenness in the surface of the osteoconductive supporting bodies.

In a further configuration of the invention, the supporting bodies are produced by an additive manufacturing process.

In a further configuration of the invention, the supporting bodies comprise calcium phosphate cement or consist of calcium phosphate cement. The calcium phosphate cement can be, in particular, a calcium phosphate cement which is subjected to a pressure, preferably absolute pressure, of at least 2 bar before complete curing. In this way, the porosity can be reduced with particular advantage.

In a further configuration of the invention, the osteoconductive supporting bodies are integrally connected to one another, in particular glued to one another.

In a further configuration of the invention, the osteoconductive supporting bodies are coated with a binder. The binder is preferably a binder which can be dissolved by heat or a solvent such as N-methyl-pyrrolidone (NMP). By using such a binder, it is possible to connect the osteoconductive supporting bodies to one another by heating and subsequent cooling or by adding a solvent. The binder can be, for example, polylactide and/or poly(lactide-co-glycolide) (PLGA).

In a further configuration of the invention, the osteoconductive supporting bodies are designed such that they favor compacting, in particular impacting, of the supporting bodies, for example by a suitable instrument such as an impactor. With regard to correspondingly suitable configurations of the osteoconductive supporting bodies, reference is made to the following statements.

In a further configuration of the invention, the osteoconductive supporting bodies are regularly shaped, i.e. are present as molded bodies. For the purposes of the present invention, the term “regularly shaped” or “molded body” shall be understood to mean in particular the shapes described below.

The osteoconductive supporting bodies, in particular molded bodies, can have a polygonal cross section. For example, the osteoconductive supporting bodies, in particular molded bodies, can have a triangular, square, rectangular, pentagonal, hexagonal, seven-cornered, octagonal, nine-corner, decagonal or star-shaped cross section.

Furthermore, the osteoconductive supporting bodies, in particular molded bodies, can have different cross sections. With regard to possible cross sections, reference is made to the cross sections mentioned in the previous paragraph.

The osteoconductive supporting bodies, in particular molded bodies, can furthermore be polyhedral, in particular cuboid, cube-shaped, tetrahedral, prism-shaped, pyramid-shaped, truncated pyramid-shaped or rod-shaped.

Furthermore, the osteoconductive supporting bodies, in particular molded bodies, can have different polyhedron shapes. In other words, the osteoconductive supporting bodies, in particular molded bodies, can be present in different polyhedron shapes. With regard to possible polyhedron shapes, reference is made to the previous paragraph.

Furthermore, the osteoconductive supporting bodies, in particular molded bodies, can have a cornerless cross section. For example, the structural elements can have an oval-shaped, in particular circular or elliptical, cross section.

Furthermore, the osteoconductive supporting bodies, in particular molded bodies, can be non-polyhedral, in particular spherical, conical, frustoconical, annular, toroidal or circular-cylindrical.

Furthermore, the osteoconductive supporting bodies, in particular molded bodies, can have different non-polyhedron shapes. In other words, the osteoconductive supporting bodies, in particular molded bodies, can be present in different non-polyhedron shapes. With regard to possible non-polyhedron shapes, reference is made to the previous paragraph.

Furthermore, the osteoconductive supporting bodies, in particular molded bodies, can be in the form of oligopods, i.e. oligopod-shaped.

The oligopods can in particular have legs that are conical and in particular rotationally symmetrical. The legs can have a cone angle of 5° to 25°, in particular 7° to 15°.

Furthermore, the oligopods can have legs with a length of 0.5 mm to 5 mm, in particular 1.5 mm to 2.5 mm.

Furthermore, the oligopods can have legs with an average diameter of 0.2 mm to 3 mm, in particular 0.3 mm to 0.7 mm.

The oligopods can also be selected from the group consisting of tripods, tetrapods, pentapods, hexapods, heptapods, octapods or mixtures thereof.

According to the invention, it can be particularly preferred if the osteoconductive supporting bodies are designed in a tetrapod shape. A tetrapod-shaped design allows a particularly effective mutual interlocking of the osteoconductive supporting bodies.

Furthermore, the osteoconductive supporting bodies, in particular molded bodies, can comprise elongated structural elements. In particular, the osteoconductive supporting bodies, in particular molded bodies, can be composed of elongated structural elements.

For the purposes of the present invention, the term “elongated structural elements” shall be understood to mean structural elements with a length-width ratio or length-diameter ratio>(pronounced: greater) 1.

The osteoconductive supporting bodies, in particular molded bodies, can preferably comprise elongated and straight structural elements. The osteoconductive supporting bodies, in particular molded bodies, are preferably composed of elongated and straight structural elements.

The elongated structural elements are preferably polyhedral, in particular cuboid, cube-shaped, prism-shaped, pyramid-shaped, truncated pyramid-shaped or rod-shaped. In other words, the structural elements of each osteoconductive supporting body, in particular molded body, preferably form a polyhedral, in particular cuboid, cube-shaped, prism-shaped, pyramid-shaped, truncated pyramid-shaped or rod-shaped arrangement.

The elongated structural elements can have a length of 0.4 mm to 5 mm, in particular 0.8 mm to 4.5 mm, preferably 1 mm to 4 mm.

Furthermore, the elongated structural elements can have a width, in particular an average width, or a diameter, in particular an average diameter, of 0.4 mm to 5 mm, in particular 0.8 mm to 4.5 mm, preferably 1 mm to 4 mm.

Furthermore, the elongated structural elements can have a cornerless cross section. For example, the structural elements can have an oval-shaped, in particular circular or elliptical, cross section.

Alternatively, the structural elements can have a polygonal cross section. For example, the structural elements can have a triangular, square, rectangular, pentagonal, hexagonal, heptagonal, octagonal, nonagonal, decagonal or star-shaped cross section.

The advantage of osteoconductive supporting bodies, in particular in the form of molded bodies, with elongated and in particular straight structural elements, in particular as described so far, is that the mutual arrangement of the structural elements per supporting body, in particular molded bodies, can create additional void volume, which improves the osteoconductive properties of the supporting body, in particular the molded body, and therefore the implant can be improved additionally. In particular, the pore size (absolute void volume) and the porosity (ratio of material volume to void volume) of human or animal bone can be optimally simulated.

In a further configuration of the invention, the osteoconductive supporting bodies are irregularly shaped.

The osteoconductive supporting bodies can be in particular in particulate form, i.e. in the form of particles.

The osteoconductive supporting bodies are preferably designed as broken material, in particular as bulk material, preferably granules.

For the purposes of the present invention, the term “bulk material” shall be understood to mean a particulate material, i.e. a material in the form of particles, the particles of which have at least one size or dimension smaller than 7 mm, preferably in a size range from 0.5 mm to 5 mm. With the at least one size or dimension can be the height and/or length and/or width (thickness) and/or the diameter, in particular average diameter, of the particles. For the purposes of the present invention, the term “granulate” shall be understood to mean an irregularly shaped, particulate material, in particular a broken and/or sieved material.

Furthermore, the osteoconductive supporting body can be designed as a non-broken material. For example, the osteoconductive supporting bodies can be designed as an additively manufactured material, i.e. as a material which is produced by an additive manufacturing process.

For the purposes of the present invention, the term “additive manufacturing process” shall be understood as a casting process for the rapid and cost-effective production of models, samples, prototypes, tools and end products, the manufacturing being carried out directly on the basis of computer-internal data models (usually transferred via an STL interface) from formless (for example liquids, gels, pastes or powders) and shape-neutral (for example band-shaped, wire-shaped or leaf-shaped) material by means of chemical and/or physical processes. Such a process can also be referred to as a generative manufacturing process.

The osteoconductive supporting bodies preferably have at least one size or dimension in a size range from 0.5 mm to 5 mm, in particular 0.1 mm to 3 mm, preferably 1 mm to 2 mm. The at least one size or dimension can be the height and/or width (thickness) and/or length and/or the diameter, in particular average diameter, of the osteoconductive supporting bodies.

In a further configuration of the invention, the osteoconductive supporting bodies are designed to be movable relative to one another, in particular displaceable relative to one another.

In a further configuration of the invention, the osteoconductive supporting bodies are designed so that they can be impacted, i.e. mutually clamped or mutually wedged.

In a further configuration of the invention, the osteoconductive supporting bodies are present in an impacted form, i.e. mutually clamped or mutually wedged.

In a further configuration of the invention, the osteoconductive supporting bodies can, preferably by means of impacting, be converted into a three-dimensional structure or matrix, in particular having voids and/or spaces, or are present in such a structure or matrix. For the purposes of the present invention such a structure or matrix can also be referred to as an osteoconductive lead structure or osteoconductive lead matrix.

The voids and/or spaces between the structure or matrix can have a diameter, in particular average diameter, of 0.1 mm to 1.2 mm, in particular 0.2 mm to 1 mm, preferably 0.3 mm to 0.8 mm.

Furthermore, with a particular advantage the structure or matrix can comprise a void volume and/or space volume of 5% to 95%, in particular 10% to 80%, preferably 20% to 70%. Such a void volume and/or space volume optimally reflects the pore volume of a human or animal cancellous bone and brings about an improvement in the osteoconductivity of the implant and in particular in the biological reconstruction of an osseous defect.

The voids and/or spaces between the structure or matrix are interconnected at least in part. In this way, the structure or the matrix optimally reflects the porosity, in particular interconnecting porosity, of the human or animal cancellous bone. In this way, ingrowth of bone tissue into a defective bone area and in particular growth of a defective bone area with vital bone tissue can also be stimulated and/or enhanced with particular advantage. This also contributes to an improvement in the osteoconductive properties of the implant and in particular the biological reconstruction of a bone defect.

It is further preferred if the structure or matrix has a modulus of elasticity, also referred to as Young's modulus, of 10 MPa to 10 GPa, in particular 50 MPa to 1 GPa, preferably 80 MPa to 350 MPa. For the purposes of the present invention the term “modulus of elasticity (Young's modulus)” shall be understood to mean the modulus of elasticity. The value of the modulus of elasticity is greater, the more resistance a material opposes to its elastic deformation. A body made of a material with a high modulus of elasticity is therefore stiffer than a body of the same configuration (same geometric dimension), which consists of a material with a low modulus of elasticity. The values for the modulus of elasticity disclosed in this paragraph optimally reflect the corresponding values of cancellous bone, which has a modulus of elasticity of 100 MPa to 1,000 MPa.

Due to the low Young's modulus described in the previous paragraph, the osteoconductive supporting bodies can be loaded mechanically in a uniform manner, i.e. homogeneous. In particular, the voids and/or spaces of the structure or matrix described in the previous paragraphs can also be mechanically loaded. By uniform or homogeneous mechanical loading of the osteoconductive supporting body and thus the implant, bone formation, in particular new bone formation, can again be achieved with particular advantage within an entire osseous defect area.

In a further configuration of the invention, the osteoconductive supporting bodies have openings or depressions, in particular through openings. As a result, the supporting bodies can be (more easily) compressed, in particular deformed, under load. Corresponding loads leading to compression of the supporting body can occur, for example, when a force is applied by a user, preferably a surgeon. As a result, compaction, in particular impaction, of the osteoconductive supporting bodies can be additionally improved, which in turn results in improved load-bearing properties of the implant.

The openings or depressions can be selected from the group consisting of holes, pores, tears, slots, cracks, gaps, notches and combinations of at least two of the openings or depressions mentioned.

The openings or depressions can also be geometrically defined or undefined openings or depressions.

In particular, the openings or depressions can have an oval, in particular circular or elliptical, cross section. Alternatively or in combination, the openings or depressions can have a polygonal, in particular triangular, square, rectangular, pentagonal, hexagonal, heptagonal, octagonal, nonagonal, decagonal or star-shaped cross section.

The openings or depressions can have a diameter of 0.01 mm to 5 mm, in particular 0.1 mm to 4 mm, preferably 0.5 mm to 3 mm. Such diameters can be preferred if the openings are designed as through openings through which, as will be explained in more detail below, a tension element is to be passed for the purpose of connecting or lashing the osteoconductive supporting bodies together.

Alternatively, the openings or depressions can have a diameter, in particular average diameter, of 60 μm to 500 μm, preferably 100 μm to 400 μm. Such diameters are preferred if the openings or depressions are designed as pores.

Preferably, the openings or depressions are pores. In other words, the osteoconductive supporting bodies can preferably be designed with open pores. In particular, the osteoconductive supporting bodies can have an interconnecting porosity.

In a further configuration of the invention, the osteoconductive supporting bodies comprise fibers. The fibers can in principle be short and/or long fibers.

For the purposes of the present invention, the term “short fibers” shall be understood to mean fibers with a length of 0.01 mm to 1 mm, in particular 0.1 mm to 1 mm, preferably 0.5 mm to 1 mm.

For the purposes of the present invention, the term “long fibers” shall be understood to mean fibers with a length>(pronounced: greater) 1 mm.

The short and/or long fibers can be metal fibers and/or polymer fibers.

In a further configuration of the invention, the implant also has a tension element. The tension element is preferably designed to be passed through through openings in the osteoconductive supporting body. This makes it particularly advantageously possible to connect the osteoconductive supporting bodies to one another or to lash them down. The tension element is therefore expediently an elongated tension element.

The tension element is preferably a textile, in particular thread-like, tension element. For example, the tension element can be a thread (tension thread), in particular a monofilament, pseudomonofilament or multifilament thread. In particular, the tension element can be a surgical suture thread.

Furthermore, the tension element can be a textile fabric, in particular in the form of a knitted, braided, crocheted, scrim, fleece or nonwoven fabric. The tension element is preferably a mesh, in particular a small-pore mesh, preferably a hernia mesh. By integrating the osteoconductive supporting bodies in a mesh-shaped tension element, a regular arrangement of the supporting bodies can be achieved.

Alternatively, the tension element can be a wire (pulling wire).

The use of a tension element enables the osteoconductive supporting body to be fastened or lashed down with particular advantage, as a result of which an immediate increase in the strength of the osteoconductive supporting bodies with one another and thus of the implant can be achieved. Such an increase in strength reduces the risk that a scaffold structure formed by the supporting bodies will break apart after a brittle fracture. Furthermore, by attaching or lashing down the osteoconductive supporting body with a particular advantage an open-pore scaffold structure can be realized. Furthermore, there is the possibility that a tension element-supporting body unit (or possibly a plurality of tension element-supporting body units) can be fixed to a further implant and/or to a bone and can thereby be fixed in place. The tensile element-supporting body unit (or tensile element-supporting body units) can be pressed onto a further implant, for example, a freshened bone, by attachment. This enables an optimal connection to the bone and the resulting pressure on the bone promotes bone growth. The force transfer at the bone defect is preferably carried out by the implant. This eliminates the pressure stimulus that stimulates the bone to build bone (stress shielding). This pressure stimulus can be built up by the tension element-supporting body unit(s) which are under pressure to the bone.

The tension element can comprise a polymer and/or metal or consist of a polymer and/or metal. The polymer can be, for example, polyethylene or polypropylene, and the metal can be, for example, titanium or tantalum.

In a further configuration of the invention, the osteoconductive supporting bodies are designed such that they can be connected to one another in a form-fitting, force-fitting and/or firmly bonded manner. The osteoconductive supporting bodies are preferably designed in such a way that they can be connected to one another in a form-fitting manner. For example, the supporting bodies can be designed such that they can be connected to one another via a plug-in system or in the manner of a plug-in system. The plug-in system can be based on a so-called pin-hole principle, preferably with an undercut for better anchoring of the osteoconductive supporting bodies. For this purpose, part of the osteoconductive supporting bodies can be provided with pins and another part of the osteoconductive supporting bodies can be provided with suitable pin holes or slots.

In a further configuration of the invention, the osteoconductive supporting bodies are connected to one another in a form-fitting, force-fitting and/or firmly bonded manner. The supporting bodies are preferably connected to one another in a form-fitting manner. For example, the osteoconductive supporting bodies can be connected to one another via a plug-in system or in the manner of a plug-in system. With regard to further features and advantages of the plug-in system, reference is made to the previous paragraph.

In a further configuration of the invention, the osteoconductive supporting bodies are designed such that they can be connected to another implant in a form-fitting, force-fitting and/or firmly bonded manner. The supporting bodies are preferably designed in such a way that they can be connected to another implant in a form-fitting manner. For example, the supporting bodies can be designed such that they can be connected to an implant via a plug-in system or in the manner of a plug-in system. The plug-in system can be based on a so-called pin-hole principle. For this purpose, the supporting bodies can be provided with a pin and the other implant can have complementary pin holes or slots. The reverse conditions can also be possible according to the invention.

In a further configuration of the invention, the osteoconductive supporting bodies are connected to one another via elongated connecting elements. For this purpose, the connecting elements preferably protrude into recesses or openings in the supporting bodies. With regard to possible configurations of the recesses or openings of the supporting bodies, reference is made to the previous statements.

In a further configuration of the invention, the osteoconductive supporting bodies have a proportion of 10% by weight to 95% by weight, in particular 20% by weight to 90% by weight, preferably 30% by weight to 70% by weight, based on the total weight of the implant.

In a further configuration of the invention, the implant is a bone replacement material.

According to a second aspect, the invention relates to a kit, preferably for the treatment and/or biological reconstruction, in particular lining and/or sealing and/or relining and/or at least partially filling, a bone defect.

The kit has the following components, spatially separated from one another:
osteoconductive supporting bodies, and
a sheath for the osteoconductive supporting bodies.

The kit is characterized in particular by the fact that the sheath comprises a sheath material or consists of a sheath material which is soluble in water or in a water-containing liquid.

The surgical kit can furthermore comprise at least one further component which is selected from the group consisting of fastening elements, artificial joint socket such as an artificial hip joint socket, further biological and/or artificial bone replacement material, tissue adhesive, a film that is not degradable in vivo or is not absorbable in vivo, a mesh that is not degradable in vivo or is not absorbable in vivo, metallic augmentation material, a fleece that is not degradable in vivo or a fleece that is not absorbable in vivo, a nonwoven fabric that is not degradable in vivo or a nonwoven fabric that is not absorbable in vivo, metallic, bendable or non-bendable lattice structure, bone cement, growth factors and one or more instruments for applying a joint socket and/or a supporting material.

The fastening elements can in particular be bone screws or nails.

The tissue adhesive can in particular be a curable adhesive composition based on cyanoacrylate monomers, in particular n-butyl-2-cyanoacrylate monomers. Such a tissue adhesive is commercially available, for example, under the trademark Histoacryl®.

By means of the film, mesh, fleece, nonwoven that are not degradable in vivo or not absorbable in vivo mentioned in connection with another kit component, or a combination thereof, in particular a composite structure, it is advantageously possible to line an open bone defect, in particular an open acetabular defect, as a result of which the prerequisites for the treatment and/or biological reconstruction of a closed bone defect, in particular a closed acetabular defect, can be created.

With regard to further features and advantages of the surgical kit, reference is made in full to the statements made for the purposes of the first aspect of the invention in order to avoid repetitions. The statements made there in particular with regard to the sheath, the sheath material and the osteoconductive supporting bodies also apply (analogously) to the surgical kit according to the second aspect of the invention.

According to a third aspect, the invention relates to a sheath for osteoconductive supporting bodies. The sheath comprises a sheath material or consists of a sheath material which is soluble in water or in a water-containing liquid.

With regard to further features and advantages of the sheath, reference is made in full to the statements made for the purposes of the first aspect of the invention in order to avoid unnecessary repetitions. The statements made there in particular with regard to the sheath, the sheath material and the osteoconductive supporting body also apply (analogously) to the sheath according to the third aspect of the invention.

According to a fourth aspect, the invention relates to the use of a material that is soluble in water or in a water-containing liquid for sheathing osteoconductive supporting bodies.

With regard to a suitable material soluble in water or in a water-containing liquid, reference is made to the soluble sheath materials disclosed in the first aspect of the invention. The soluble sheath materials described there can also be considered as materials for the use according to the fourth aspect of the invention.

With regard to further features and advantages of the use, reference is made in full to the statements made for the purposes of the first aspect of the invention in order to avoid unnecessary repetitions. The statements made there in particular with regard to the sheath and the osteoconductive supporting bodies also apply (analogously) to the use according to the fourth aspect of the invention.

According to a fifth aspect, the invention relates to a method for the treatment and/or biological reconstruction, in particular lining and/or sealing and/or relining and/or at least partially filling, a bone defect.

The bone defect is preferably an acetabular defect, in particular a closed or open acetabular defect.

The method comprises the following step:
Placing an implant according to the first aspect of the invention in the bone defect.

In a configuration of the invention, the method also comprises the following step:
Loading the placed implant by means of an impactor, i.e. a surgical instrument for compacting, in particular impacting, the osteoconductive supporting bodies.

In a further configuration of the invention, the method also comprises the following step:
Applying a bone cement to the placed, in particular loaded, implant.

In a further configuration of the invention, the method also comprises the following step:
Applying an artificial joint socket, in particular an artificial hip joint socket, to the sheath and optionally fixing/attaching the artificial joint socket, in particular an artificial hip joint socket, to the sheath.

In a further configuration of the invention, the method also comprises the following step:
Attaching the artificial joint socket, in particular an artificial hip joint socket, to a bone, preferably to a bone that at least partially surrounds the bone defect.

With regard to further features and advantages of the method, reference is also made in full to the statements made for the purposes of the first aspect of the invention in order to avoid repetition. The statements made there with respect to the implant, in particular the sheath, the sheath material and the osteoconductive supporting bodies, also apply (analogously) to the method according to the fifth aspect of the invention.

### DETAILED DESCRIPTION

Further features and advantages of the invention result from the following description of preferred embodiments with the aid of an exemplary embodiment. Features of the invention can be realized individually or in combination with one another. The exemplary embodiments described below serve to further explain the invention without limiting it thereto.

### Exemplary Embodiment

1. Materials:

Carboxymethyl cellulose (Tylopur C), polyvinyl alcohol (Mowiol 56-98, high molar), polyvinyl alcohol (Mowiol 4-98, low molar), water for injections (WVI).

2. Execution:

First, a 20% low molar PVA solution and a 20% high molar PVA solution were made in a Schott flask. For this purpose, 20 g of low molecular weight polyvinyl alcohol and 80 g of water for injection as well as 20 g of high molecular weight polyvinyl alcohol and 80 g of water for injection were filled into separate Schott flasks. The two mixtures were then stored in a warming cabinet at 95° C. for 24 hours until they were completely dissolved. Then a 3% carboxymethyl cellulose solution (3 g sodium carboxymethyl cellulose+97 g water for injection) was prepared in a laboratory mixer (laboratory mixer model ESCO type EL 10) or homogenizer under vacuum (−0.8 bar) at 35° C. and for a mixing time of about 3 to 4 hours.

A mixture of the low-molar PVA solution and the high-molar PVA solution, a mixture of the low-molar PVA solution and the carboxymethyl cellulose solution and a mixture of the high-molar PVA solution and the carboxymethyl cellulose were then prepared. These mixtures were each prepared using the laboratory mixer or homogenizer mentioned above (maximum temperature 35° C., vacuum −0.8 bar, mixing time at least 2 hours).

The mixtures prepared were then formed into a film with the aid of a squeegee using a laboratory dryer and fixer (Mathis type LTF 143691) (at approx. 55° C. to 60° C. and during a drying time of approx. 25 minutes).

The thickness of the films was 38 μm to 45 μm. The films dissolved in water within 20 s to 40 s.

Depending on the setting of the gap size, films could be made in different thicknesses. The thicker the films, the more slowly they dissolve in water.

Films prepared with a thickness of 50 μm to 65 μm dissolved in water within approx. 3 minutes to 5 minutes.

Using a bar welder the films prepared could be successfully welded to form a small bag, which was previously filled with osteoconductive supporting bodies.

As an alternative to a welding process, it is conceivable to seal the foils by means of a tissue adhesive, such as, for example, a tissue adhesive based on n-butyl-2-cyanoacrylate monomers sold under the registered trademark Histoacryl®.

1. An implant for the treatment and/or biological reconstruction of a bone defect, comprising:
osteoconductive supporting bodies; and
a sheath that at least partially surrounds the osteoconductive supporting bodies, the sheath comprising a sheath material that is soluble in water or in a water-containing liquid.

2. The implant according to claim 1, wherein the sheath material comprises starch, amylose, amylopectin, dextran, methyl cellulose, hydroxymethyl cellulose, ethyl cellulose, hydroxyethyl cellulose, propyl cellulose, hydroxypropyl cellulose, butyl cellulose, hydroxybutyl cellulose, hydroxyethylmethyl cellulose, hydroxypropylmethyl cellulose, carboxymethyl cellulose, hyaluronic acid, chondroitin-4-sulfate, chondroitin-6-sulfate, keratin sulfate, alginic acid, heparin, heparan sulfate, chitosan, salts thereof or mixtures thereof.

3. The implant according to claim 1, wherein the sheath material comprises polyvinyl alcohol, polyethylene glycol, ethylene oxide-propylene oxide copolymers (EO-PO copolymers), ethylene oxide-propylene oxide block copolymers (EO-PO block copolymers), acrylic acid homopolymers, acrylic acid copolymers, polyvinylpyrrolidone homopolymers, polyvinylpyrrolidone copolymers or mixtures thereof.

4. The implant according to claim 1, wherein the sheath has a first section and a second section that are designed differently.

5. The implant according to claim 4, wherein the first and second sections differ from one another with regard to the solubility of the sheath in water or in a water-containing liquid and/or with regard to the thickness of the sheath and/or with regard to the structure of the sheath.

6. The implant according to claim 4, wherein the first and second sections differ from one another with regard to chemical composition.

7. The implant according to claim 4, wherein the first section comprises a sheath material that is more soluble in water or in a water-containing liquid than the second section.

8. The implant according to claim 4, wherein the first section comprises a less cross-linked sheath material that is soluble in water or in a water-containing liquid than the second section.

9. The implant according to claim 4, wherein the first section comprises a non-cross-linked sheath material that is soluble in water or in a water-containing liquid, and the second section comprises a cross-linked sheath material that is soluble in water or in a water-containing liquid.

10. The implant according to claim 4, wherein the first section comprises a higher proportion of a sheath material that is soluble in water or in a water-containing liquid than the second section.

11. The implant according to claim 4, wherein the first section comprises a sheath material that is soluble in water or in a water-containing liquid, and the second section comprises a sheath material that is insoluble in water or in a water-containing liquid.

12. The implant according to claim 4, wherein the first section has a smaller thickness than the second section.

13. The implant according to claim 4, wherein the first section is non-textile, and the second section is textile.

14. The implant according to claim 4, wherein the first section is configured to face a bone in the implanted state, and the second section is configured to face an artificial socket in the implanted state.

15. The implant according to claim 4, wherein the osteoconductive supporting bodies are present in isolated form and comprise apatite, tricalcium phosphate, and/or biphasic calcium phosphate.

16. The implant according to claim 15, wherein the osteoconductive supporting bodies have a porosity of 1% to 50%.

17. The implant according to claim 15, wherein the osteoconductive supporting bodies are non-porous.
